(19) United States
(12) Patent Application Publication
(10) Pub. No.: US 2020/0085784 A1
(43) Pub. Date: Mar. 19, 2020

(54) METHODS TO TREAT FIBROSIS, NASH, AND NAFLD

(71) Applicant: Minneamrita Therapeutics, LLC, Tampa, FL (US)

(72) Inventors: Ashok K. Saluja, Pinecrest, FL (US); Vikas Dudeja, Pinecrest, FL (US); Shweta Lavania, Coral Gables, FL (US); Aniket Nikam, Miami, FL (US); Sulagna Banerjee, Miami, FL (US)

(73) Assignee: Minneamrita Therapeutics, LLC, Tampa, FL (US)

(21) Appl. No.: 16/569,450

(22) Filed: Sep. 12, 2019

Related U.S. Application Data
(60) Provisional application No. 62/730,924, filed on Sep. 13, 2018.

(51) Int. Cl. A61K 31/365; A61K 38/26; A61K 31/192; A61K 45/06; A61P 1/16

(52) U.S. Cl. CPC A61K 31/365; A61K 38/26; A61P 1/16; A61K 45/06; A61K 31/192

(57) ABSTRACT

The invention provides a method for treating fibrosis, nonalcoholic fatty liver disease (NAFLD) or nonalcoholic steatohepatitis (NASH) in an animal, comprising administering to the animal, a compound of formula I:

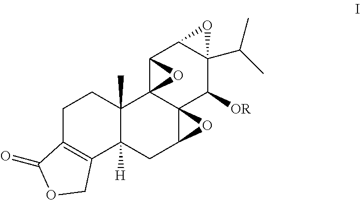

or a pharmaceutically acceptable salt thereof.

### CROSS-REFERENCE TO RELATED APPLICATIONS

This application claims priority to U.S. Provisional Application No. 62/730,924 that was filed on Sep. 13, 2018. The entire content of the application referenced above is hereby incorporated by reference herein.

### BACKGROUND

Triptolide is a naturally occurring compound obtained from the plant Tripterygium wilfordii. Triptolide is known to be useful in treating autoimmune diseases, transplantation rejection (immunosuppression), and possesses anticancer and anti-fertility effects as well as other biological effects (Qui and Kao, 2003, Drugs R. D. 4, 1-18). Triptolide has strong antitumor effects against xenograft tumors (for example, Yang et al. Mol. Cancer Ther, 2003, 2, 65-72). Triptolide is an anti-apoptotic agent with multiple cellular targets that are implicated in cancer growth and metastasis. Triptolide inhibits NF-kB activation, induces bid cleavage, blocks induction of the survival gene p21 WAF1/^{Cip1} (Wang et al. Journal of Molecular Medicine, 2006, 84, 405-415) and inhibits the function of heat shock transcription factor 1 (HSF1) thereby suppressing endogenous HSP70 gene expression (Westerheide et al. 2006, Journal of Biological Chemistry, 281, 9616-9622). Triptolide also functions as a potent tumor angiogenesis inhibitor (He et al. 2010, Int. Journal of Cancer, 126, 266-278).

International Patent Application Publication Number WO2010/129918 reports triptolide prodrugs that are reported to be useful for treating cancer. One of these compounds, Minnelide® (14-O-phosphonooxymethyltriptolide disodium salt), is in clinical development for the treatment of various cancers. Fibrogenesis is a complex wound-healing process that requires the interaction of several cell types that become triggered by a broad spectrum of cytokines, chemokines, and nonpeptide mediators including reactive oxygen species, lipid mediators, and hormones. Progressive fibrosis is linked to architectural changes of the liver with increased stiffness favoring portal hypertension, it may advance to end-stage cirrhosis, and it provides a microenvironment that predisposes to liver cancer. Consequently, the presence of liver fibrosis in biopsy samples is the strongest predictor of liver-related complications and mortality in patients with nonalcoholic fatty liver.

Liver fibrosis remains a major health problem, as fibrotic liver diseases have a high mortality rate and predispose to liver failure. A better understanding of the mechanisms associated in the initiation, progression, and resolution of fibrosis is crucially needed.

Antifibrotic agents are specifically needed for the prevention of progression and the induction of reversal of advanced alcoholic (ASH) and non-alcoholic steatohepatitis (NASH), viral hepatitis B and C—despite the advent of highly effective antiviral therapies—, and of (pediatric) metabolic, biliary and autoimmune liver diseases.

Liver fibrosis remains a major health problem as fibrotic liver diseases have a high mortality rate and predispose to liver failure. Although intense research during the last 20 years has led to considerable improvements in the understanding of liver fibrosis pathogenesis, effective antifibrotic therapies are still lacking. To date, no general antifibrotic therapy is currently available in clinical practice, leaving treatment of the underlying disease and ultimately liver transplantation as the only therapeutic options for advanced liver fibrosis. Moreover, the current options for the treatment of fibrotic diseases are extremely limited, and to date no effective drug has emerged that successfully targets established fibrosis. At present, most of the antifibrotic agents are currently tested in patients with nonalcoholic fatty liver diseases which results in additional metabolic effects. Thus it is unclear whether the expected results from the ongoing trials can be extrapolated to other chronic liver diseases such as cirrhosis or early stages of liver fibrosis.

Non-alcoholic fatty liver disease (NAFLD) presents a substantial health burden in modern society with increasing incidence not only in western countries but worldwide. NAFLD is considered as one of the most common cause of chronic liver disease (CLD). The key risks factors for NAFLD include excess body weight, insulin resistance, type 2 diabetes (T2D), hypertension, decreased high-density lipoproteins (HDL) and hypertriglyceridemia. NAFLD starts as relatively benign steatosis, which is reversible and mainly characterized by hepatic fat deposition. It covers a spectrum of liver damage ranging from simple steatosis to nonalcoholic steatohepatitis (NASH), fibrosis, and cirrhosis. Progression of steatosis to NASH is a severe life-threatening disease. Subsequently, if NASH progresses to cirrhosis or hepatocellular carcinoma, it causes a serious health issue. Of interest, the patients who are exposed to the same risk factors for metabolic diseases, (obesity, and type-II diabetes) does not always develop NASH, the reasons for which are still unknown. Studies have shown NAFLD to be the most common form of chronic liver disease with an incidence of 10-24% in the U.S., and perhaps similar statistics in Europe and Asia. Also, the incidence for NASH is about 3-5% of the lean and 19% of obese population. NASH defines a subgroup of nonalcoholic fatty liver disease where liver steatosis coincides with hepatic cell injury involving apoptosis and hepatocyte ballooning along with inflammation. To date, no pharmacological treatment is approved for NAFLD/NASH.

Currently there is a need for improved therapeutics for treating fibrosis (e.g. fibrosis in the liver or lung), as well improved therapeutics for treating nonalcoholic fatty liver disease (NAFLD) or nonalcoholic steatohepatitis (NASH). In particular, there is a need for novel and effective anti-fibrotic agents which are effective in reversing the early stages of fibrosis.

### SUMMARY

Applicant has determined that Minnelide™ (14-O-phosphonooxymethyltriptolide disodium salt) is useful for treating fibrosis, nonalcoholic fatty liver disease (NAFLD), and nonalcoholic steatohepatitis (NASH). Accordingly, in one embodiment, the invention provides a method for treating fibrosis, nonalcoholic fatty liver disease (NAFLD) or nonalcoholic steatohepatitis (NASH) in an animal, comprising administering to the animal, a compound of formula I:

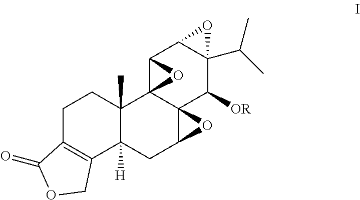

wherein:

R is H or (CR¹R²O)ₙP(O)(OH)₂;

each R¹ is independently H, (C₁-C₆)alkyl, aryl(C₁-C₆)alkyl-, (C₃-C₆)cycloalkyl or aryl; and each R² is independently H, (C₁-C₆)alkyl, aryl(C₁-C₆)alkyl-, (C₃-C₆)cycloalkyl or aryl; or R¹ and R² together with the atom to which they are attached form a (C₃-C₇)cycloalkyl; wherein any alkyl or cycloalkyl of R¹ or R² may be optionally substituted with one or more (e.g. 1, 2, 3, 4 or 5) groups selected from halo, (C₁-C₆)alkoxy and NR^{a}R^{b} and wherein any aryl of R¹ or R² may be optionally substituted with one or more (e.g. 1, 2, 3, 4 or 5) groups selected from halo, (C₁-C₆)alkyl, (C₁-C₆)alkoxy, NR^{a}R^{b}, nitro and cyano;

R^{a} and R^{b} are each independently selected from H, (C₁-C₆)alkyl, (C₃-C₆)cycloalkyl and aryl; or R^{a} and R^{b} together with the nitrogen to which they are attached form a pyrrolidino, piperidino, piperazino, azetidino, morpholino, or thiomorpholino; and

n is 1, 2 or 3;

or a pharmaceutically acceptable salt thereof.

The invention also provides a compound of formula I, or a pharmaceutically acceptable salt thereof for use in the prophylactic or therapeutic treatment of fibrosis, nonalcoholic fatty liver disease (NAFLD) or nonalcoholic steatohepatitis (NASH).

The invention also provides the use of a compound of formula I, or a pharmaceutically acceptable salt thereof for the manufacture of a medicament for the treatment of fibrosis, nonalcoholic fatty liver disease (NAFLD) or nonalcoholic steatohepatitis (NASH) in a mammal (e.g. a human).

As shown in the Examples below, a representative compound of formula I, Minnelide™ (14-O-phosphonooxymethyltriptolide disodium salt) has been shown to provide promising results in two models of liver fibrosis.

### BRIEF DESCRIPTION OF THE FIGURES

FIG. 1. Efficacy of Minnelide alone and in combination with Elafibranor or Liraglutide using DIO-NASH mouse model; and Reference Study section of FIG. 1 refers to a historical reference study (published as Tølbøl, et al. World J Gastroenterol. Jan. 14, 2018; 24(2): 179-194) on efficacy of Elafibranor alone or Liraglutide alone using DIO-NASH mouse model.

FIG. 2A. Histological quantitative assessment of liver Galectin-3. Scanned slides were analyzed for crude detection of tissue at low magnification first.

FIG. 2B. Representative images of liver stained with anti-Galectin-3 at high magnification (upper insert); and detection of steatosis (white), Galectin-3 (gray) and tissue (black) (lower insert); the liver Galectin-3 fraction was estimated as % of total tissue.

FIG. 2C. Representative images of liver stained with anti-Galectin-3 (Biolegend, cat. 125402) at termination (magnification 20×, scale bar=100 μm).

FIG. 2D. Terminal relative liver Galectin-3 (Gal-3) quantified by morphometry (% fractional area). Values expressed as mean of n=12-14+SEM. Dunnett's test one-factor linear model. **: P<0.01, ***: P<0.001 compared to Vehicle+Vehicle.

FIG. 3. Terminal relative liver hydroxyproline (HP) quantified by morphometry (% fractional area). Values expressed as mean of n=12-14+SEM. Dunnett's test one-factor linear model. **: P<0.01, ***: P<0.001 compared to Vehicle+Vehicle.

FIG. 4. Terminal relative liver α-SMA quantified by morphometry (% fractional area). Values expressed as mean of n=12-14+SEM. Dunnett's test one-factor linear model. **: P<0.01, ***: P<0.001 compared to Vehicle+Vehicle.

### DETAILED DESCRIPTION

### Definitions

The term “(C₁-C₆)alkyl” as used herein refers to alkyl groups having from 1 to 6 carbon atoms which are straight or branched groups. This term is exemplified by groups such as methyl, ethyl, n-propyl, iso-propyl, n-butyl, t-butyl, isobutyl, n-pentyl, neopentyl, and n-hexyl, and the like.

The term “(C₁-C₆)alkoxy” as used herein refers to the group (C₁-C₆)alkylO— wherein (C₁-C₆)alkyl is as defined herein. This term is exemplified by groups such as methoxy, ethoxy, propoxy, isopropoxy, butoxy, iso-butoxy, sec-butoxy, pentoxy, 3-pentoxy, or hexyloxy, and the like.

The term “(C₃-C₇)cycloalkyl” as used herein refers to a saturated or partially unsaturated cyclic hydrocarbon ring system comprising 3 to 7 carbon atoms. This term is exemplified by such groups as cyclopropyl, cyclobutyl, cyclopentyl, cyclohexyl, cyclohexene, or cycloheptane, and the like.

The term “aryl” as used herein refers to a phenyl radical or an ortho-fused bicyclic carbocyclic radical having about nine to ten carbon ring atoms in which at least one ring is aromatic. This term is exemplified by such groups phenyl, indanyl, indenyl, naphthyl, 1,2-dihydronaphthyl and 1,2,3,4-tetrahydronaphthyl.

The term “aryl(C₁-C₆)alkyl-” as used herein refers to the group aryl-(C₁-C₆)alkyl- wherein (C₁-C₆)alkyl and aryl are as defined herein. This term is exemplified by such groups as benzyl and phenethyl and the like.

As used herein, the term “comprising” means the elements recited, or their equivalent in structure or function, plus any other element(s) which are not recited. The terms “having” and “including” are also to be construed as open ended unless the context suggests otherwise. Terms such as “about,” “generally,” “substantially,” and the like are to be construed as modifying a term or value such that it is not an absolute, but does not read on the prior art. Such terms will be defined by the circumstances and the terms that they modify are understood by those of skill in the art. This includes at the very least the degree of expected experimental error, technique error, and instrument error for a given technique used to measure a value.

The phrases “therapeutically effective amount” and “pharmaceutically effective amount” are used herein, for example, to mean an amount sufficient to reduce or inhibit in vivo cancerous cell growth upon administration to a living mammal. The phrases are meant to refer to the amount determined to be required to produce the physiological effect intended and associated with the given active ingredient, as measured according to established pharmacokinetic methods and techniques, for the given administration route.

The phrase “inhibitory effective amount” as used in association with the amount of active compound and composition is meant to refer, for example, to exhibited antitumor properties as demonstrated using standard cell culture assay techniques.

As used herein, the term “prodrug” is meant to refer to a pharmaceutical compound that requires further metabolism (including but not limited to the liver) before becoming biologically active.

It will be appreciated by those skilled in the art that compounds having a chiral center may exist in and be isolated in optically active and racemic forms. Some compounds may exhibit polymorphism. It is to be understood that the compounds encompasse any racemic, optically-active, polymorphic, or stereoisomeric form, or mixtures thereof, of a compound, which possess the useful properties described herein, it being well known in the art how to prepare optically active forms, for example, by resolution of the racemic form by recrystallization techniques, by synthesis from optically-active starting materials, by chiral synthesis, or by chromatographic separation using a chiral stationary phase.

A salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts and inorganic salts.

The term “organic cation or inorganic cation” or “cationic organic or inorganic salt” include organic cations or inorganic cations (e.g. metal or amine salts) that are well known in the art and include cationic moieties that can form an ionic association with the O moieties on the compound and not significantly adversely affecting the desired properties of the prodrug for purposes of the invention. The term “pharmaceutically acceptable organic cations or inorganic cations” or “pharmaceutically acceptable cationic organic or inorganic salt” include the “organic cations or inorganic cations” which are pharmaceutically acceptable for use in a mammal and are well known in the art.

Organic cations or inorganic cations include but are not limited to lithium, sodium, potassium, magnesium, calcium, barium, zinc, aluminium and amine cations. Amine cations include but are not limited to cations derived from ammonia, triethylamine, tromethamine (TRIS), triethanolamine, ethylenediamine, glucamine, N-methylglucamine, glycine, lysine, ornithine, arginine, ethanolamine, choline and the like. In one embodiment, the amine cations are cations wherein X⁺ is of the formula YH⁺ wherein Y is ammonia, triethylamine, tromethamine (TRIS), triethanolamine, ethylenediamine, glucamine, N-methylglucamine, glycine, lysine, ornithine, arginine, ethanolamine, choline and the like.

In one embodiment suitable cationic organic or inorganic salts that can be used include cationic moieties that can form an ionic association with the O moieties on the compound and not significantly adversely affecting the desired properties of the prodrug for purposes of the invention, e.g., increased solubility, stability, and rapid hydrolytic release of the active compound form. Preferably, X is selected from Li⁺, K⁺, or Na⁺. More preferably, X is Na⁺ thus forming the disodium salt.

Pharmaceutically acceptable salts can also include salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts. Salts, including pharmaceutically acceptable salts may be obtained using standard procedures well known in the art, for example by reacting a sufficiently basic compound such as an amine with a suitable acid affording a physiologically acceptable anion.

The compounds of formula I include the free acids (e.g. —OP(O)(OH)₂), mono-salts (e.g. —OP(O)(OH)(O⁻X⁺)) and di-salts (e.g. —OP(O) O⁻X⁺)₂). The acid and the salts may be purified by a variety of techniques well known in the art such as chromatography, followed by lyophilization or recrystallization.

It will be appreciated by those skilled in the art that a compound of formula I wherein X⁺ is an organic cation or inorganic cation can be converted to a compound of formula I comprising one or more different organic or inorganic cations. Such a conversion can be accomplished using a variety of well known techniques and materials including but not limited to ion exchange resins, ion exchange chromatography and selective crystallization.

### EMBODIMENTS

A specific value for R¹ is H or (C₁-C₆)alkyl.

Another specific value for R¹ is H.

Another specific value for R¹ is (C₁-C₆)alkyl.

Another specific value for R¹ is methyl or ethyl.

A specific value for R² is H or (C₁-C₆)alkyl.

Another specific value R² is H.

A specific value for X⁺ is H.

Another specific value X⁺ is independently lithium, sodium, potassium, magnesium, calcium, barium, zinc or aluminium.

Another specific group of compounds of formula I are compounds wherein X⁺ is of the formula HY⁺ wherein Y is independently ammonia, triethylamine, tromethamine, triethanolamine, ethylenediamine, glucamine, N-methylglucamine, glycine, lysine, ornithine, arginine, ethanolamine or choline.

Another specific value for X⁺ is independently Li⁺, K⁺ or Na⁺.

Another specific value for X⁺ is Na⁺.

A specific compound of formula I is 4-O-phosphonooxymethyltriptolide disodium salt, 14-O-phosphonooxyethyltriptolide disodium salt or 14-O-phosphonooxypropyltriptolide disodium salt, or a salt thereof.

A specific group of salts are salts of formula Ia:

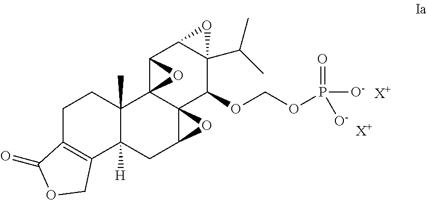

wherein each X⁺ is independently a pharmaceutically acceptable cationic organic or inorganic salt.

Processes that can be used to prepare compounds of formula I and intermediates useful for preparing compounds of formula 1 are shown in Scheme 1 and Scheme 2. The compounds and salts can also be prepared as described in International Patent Application Publication Number WO2010/129918.

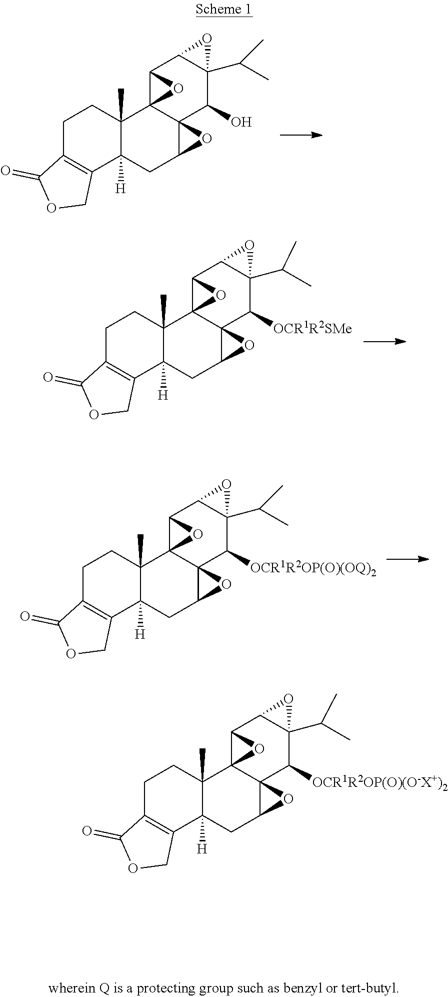

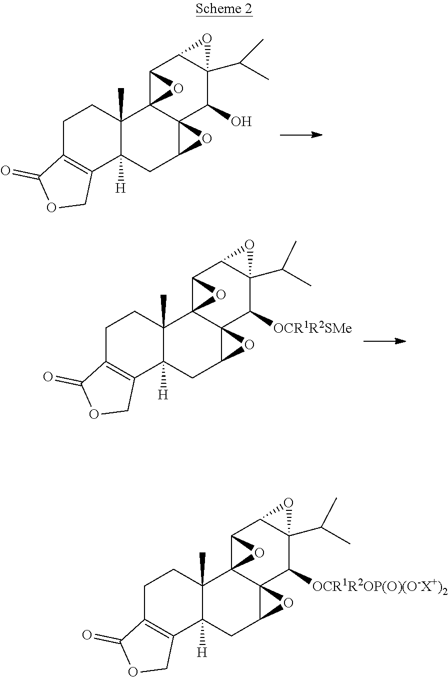

A compound of formula I can be prepared by removing one or more protecting groups from a compound of formula IA:

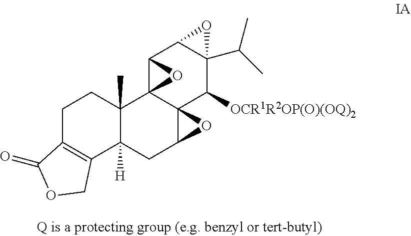

to provide the corresponding compound of formula I. Thus, the intermediate of formula IA is useful for preparing a compound of formula I.

A compound of formula I can also prepared by converting the —SMe group from a compound of formula IB:

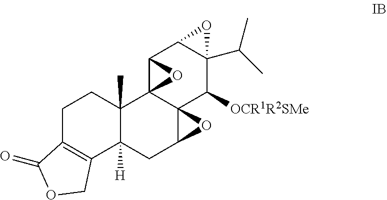

to a —OP(O)(O⁻X⁺)₂ group to provide the corresponding compound of formula I. Thus, the intermediate of formula IB is useful for preparing a compound of formula I.

A compound of formula I can also be prepared by removing one or more protecting groups from a compound of formula IC:

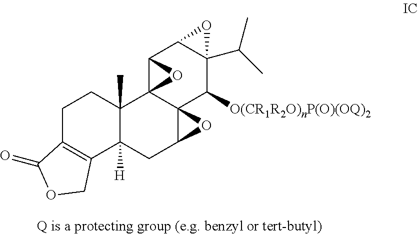

to provide the corresponding compound of formula I. Thus, the intermediate of formula IC is useful for preparing a compound of formula I.

A compound of formula I can also prepared by converting the —SMe group from a compound of formula ID:

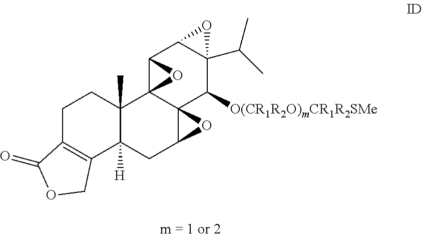

to a —OP(O)(O⁻X⁺)₂ group to provide the corresponding compound of formula I. Thus, the intermediate of formula ID is useful for preparing a compound of formula I.

The compound of formula I or the salt thereof can be formulated into pharmaceutical compositions as well by combining together with a pharmaceutically acceptable carrier. Pharmaceutical compositions can be prepared in accordance with well-known compounds and techniques readily available to those skilled in the pharmaceutical field. For purposes of the invention, the pharmaceutically acceptable carrier can be any conventional and readily available biologically compatible or inert substance which is chemically compatible with the active pharmaceutical ingredient and does not significantly attenuate its intended therapeutic effect upon formulation or delivery. Pharmaceutically acceptable salts can be prepared using standard procedures and techniques well known in the art.

The solid form of a compound of formula I or the salt thereof can be a nanoparticle and thus formulated as a nanoparticle. The compound of formula I or the salt thereof can be formulated using a variety of excipient formulations and prepared in various dosage forms as described below. The chemical properties and attributes associated with the compounds can afford the preparation of an oral solid dosage forms.

The compound of formula I or the salt thereof can be formulated as pharmaceutical compositions and administered to a recipient in a variety of forms suitable for the desired particular administration route or system. Administration routes can include but are limited to oral routes, parenteral routes, intravenous routes (including intravenous routes by pump injection), intramuscular routes, topical routes including eye drops, subcutaneous routes and mucosal routes. Compounds can be administered systemically, e.g. orally, in combination with a pharmaceutically acceptable carrier such as an inert diluent or assimilable edible carrier. Thus the pharmaceutical composition comprising the compound as the active ingredient can be prepared in a variety of dosage forms. For example, the compositions can be encapsulated in hard or soft capsules (e.g., gelatin or vegetable-derived capsular materials). The compositions can be compressed into ingestible or transmucosal tablet form, troches, capsules, elixirs, suspensions, syrups, wafers, suppositories and the like. The amount of active ingredient can vary according to the specific desired pharmaceutically effective dosage amount.

Tablets, troches, pills, capsules, and the like can contain additional ingredients such as binders (such as gum tragacanth, acacia, corn starch or gelatin); excipients such as dicalcium phosphate; disintegrants such as corn starch, potato starch, alginic acid, and the like; lubricants (such as magnesium stearate) which can be used for tablet compression techniques, for example; sweeteners such as sucrose, fructose, lactose or aspartame; and flavoring agents such as peppermint, wintergreen, cherry, and the like. Additional ingredients which may be included in compositions are mannitol, urea, dextranes, and lactose non-reducing sugars.

When the dosage form is a capsule, it can contain a liquid carrier including polyethylene glycol, vegetable oil, etc. Other materials that can be used with certain dosage forms include gelatin, wax, shellac, sugar, and the like. Syrups or elixir forms can contain sucrose, fructose as sweeteners, methyl and propylparabens as preservatives, dyes and colorants, and flavoring agents.

When administered intravenously or intraperitoneally by infusion or injection, solutions of the active ingredient or its salts can be prepared in, for example, water or saline optionally containing a non-toxic surfactant. Dispersions can be prepared in glycerol, liquid polyethylene glycols, triacetin, and mixtures thereof and in oils. Storage conditions may necessitate the inclusion of a preservative as well.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Injectible or infusible pharmaceutical dosage forms can include sterile aqueous solutions or dispersions or sterile powders comprising the active compounds prepared for extemporaneous formulation. Liquid carriers can include solvents or liquid dispersion mediums comprising water, ethanol, a polyol (e.g., glycerol, propylene glycol, polyethylene glycols), and the like. Various agents can be added to inhibit or prevent antimicrobial activity, such as parabens, chlorobutanol, phenol, sorbic acid, thimerosal, and the like.

Compounds and compositions can be administered as a single dose or in multiple dose intervals. The dosage amount, dosage form, route of administration, and the particular formulation ingredients can vary corresponding to the desired plasma concentration and pharmacokinetics involved.

Useful dosages of the compounds can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds can also be administered in combination with other therapeutic agents, for example, other agents that are useful for the treatment of fibrosis, nonalcoholic fatty liver disease (NAFLD) or nonalcoholic steatohepatitis (NASH). Examples of such therapeutic agents include: insulin sensitizing agents (e.g. metformin), thiazolidineones (e.g. pioglitazone or rosiglitazone), vitamin E, ursodeoxycholic acid, omega-3 fatty acids, galectin-3 inhibitors (e.g., GR-MD-02), and statins. See N. Chalasani, et al., Hepatology, 2012, 55, 9, 2005-2023. Accordingly, in one embodiment the invention also provides a composition comprising a compound of formula I, or a pharmaceutically acceptable salt thereof, a therapeutic agent, and a pharmaceutically acceptable diluent or carrier. The invention also provides a kit comprising a compound of formula I, or a pharmaceutically acceptable salt thereof, a therapeutic agent, packaging material, and instructions for administering the compound of formula I or the pharmaceutically acceptable salt thereof and the therapeutic agent to an animal (e.g. mammal) to treat fibrosis, nonalcoholic fatty liver disease (NAFLD) or nonalcoholic steatohepatitis (NASH). In one embodiment, the therapeutic agent is selected from the group consisting of insulin sensitizing agents (e.g. metformin), thiazolidineones (e.g. pioglitazone and rosiglitazone), vitamin E, ursodeoxycholic acid, omega-3 fatty acids, galectin-3 inhibitors (e.g., GR-MD-02), and statins.

In one embodiment, the therapeutic agent is a GLP-1 agonist. GLP-1 agonists mimic the actions of the glucagon-like peptide. By activating GLP-1 receptors, GLP-1 agonists and endogenous GLP-1 can reduce blood glucose levels and help T2DM patients reach glycernic control. In one embodiment, the therapeutic agent is selected from the group consisting of Albiglutide (Tanzeum), Dulaglutide (Trulicity), Exenatide (Byetta), Extended-release exenatide (Bydureon), Liraglutide (Victoza), Lixisenatide (Adlyxin) and Semaglutide (Ozempic). In one embodiment, the therapeutic agent is liraglutide.

In one embodiment, the therapeutic agent is a PPAR agonist. PPAR agonists act on the peroxisome proliferator-activated receptor. In one embodiment, the therapeutic agent is a pan PPAR agonist. In one embodiment, the therapeutic agent is a PPARα/δ agonist. In one embodiment, the therapeutic agent is a PPARγ/δ agonist. In one embodiment, the therapeutic agent is a PPARα agonist. In one embodiment, the therapeutic agent is a PPARδ agonist. In one embodiment, the therapeutic agent is a PPARγ agonist. In one embodiment, the therapeutic agent is selected from the group consisting of Albiglutide (Tanzeum), Dulaglutide (Trulicity), Elafibranor, Exenatide (Byetta), extended-release exenatide (Bydureon), Liraglutide (Victoza), Lixisenatide (Adlyxin), elafibranor (GFT505), and Semaglutide (Ozempic). In one embodiment, the therapeutic agent is elafibranor or a salt thereof.

The invention will now be illustrated by the following non-limiting Examples.

### EXAMPLES

### Example 1 Mouse Models of Fibrosis

Two animal models were used: Mouse model of Carbon tetrachloride (CCl₄). In this model, C57Bl6/J mice (8 weeks of age; ˜25 g) received twice a week during 4 weeks 250 μL i.p. of either olive oil or CCl₄ at a dose of 3.5 ml/kg diluted in olive oil. Animals were treated with 0.2 mg/kg Minnelide after 4 weeks of CCl₄.

Mouse model of CCl₄+Diethylnitrosamine (DEN)-induced liver injury. C57Bl6/J mice (3 weeks of age; ˜15 g) received one single dose of DEN (25 mg/kg). From 8 weeks of age; (˜25 g) they receive twice a week during 4 weeks 250 μL i.p. of either olive oil or CCl4 at a dose of 0.2 ml/kg diluted in olive oil. Animals were treated with 0.2 mg/kg Minnelide after 4 weeks of CCl₄.

For both models, injury and fibrosis were determined by examination of histological specimens by H&E staining as well as picrosirius red staining.

### Results

Minnelide Prevented and Reversed Liver Fibrosis in CCl₄ and DEN+CCl₄ Mouse Models.

Minnelide at 0.2 mg/kg inhibited CCl₄ and DEN+CCl₄-induced collagen deposition compared to the vehicle, as observed by Massons Trichrome and Sirius red staining. Histologic examination of the liver H&E staining indicated that Minnelide reduced steatosis, ballooning, and inflammatory foci induced by the CCl₄ and DEN+CCl₄ administration at the 8 week time point. Minnelide also reduced α-SMA expression assessed by immunofluorescence staining.

Minnelide Inhibited Fibrotic Gene Expression in CCl₄ and DEN+CCl₄ Mouse Models.

Minnelide at 0.2 mg/kg inhibited CCl₄ and DEN+CCl₄-induced inhibited the expression of the key fibrotic genes overexpression such as α-SMA, collagen1, and fibronectinalong with TGF-β1, TGF-β2, TGF-β3 (FIG. 14); TGF-β receptors TGF-βR1 and TGF-βR2.

Minnelide Inhibited Inflammation Associated Gene Expression in DEN+CCl₄ Mouse Models.

Minnelide decreased the expression of Tnf-α, IL6, IL-1β and iNOS at the 8 week time point. Minnelide inhibited Inflammasome-related gene expression in DEN+CCl₄ mouse models. Minnelide decreased the expression of the inflammasome genes NOD-like receptor family, pyrin domain containing 3 (NLRP3), apoptosis-associated speck-like protein containing a CARD, caspase1, interleukin (IL)-1β, and IL18 at the 8 week time point.

Minnelide Prevented and Reversed Liver Fibrosis in DEN+CCl₄ Mouse Models.

Minnelide at 0.2 mg/kg inhibited DEN+CCl₄-induced collagen deposition compared to the vehicle, as observed by Massons Trichrome and Sirius red staining. Histologic examination of the liver H&E staining indicated that Minnelide reduced steatosis, ballooning, and inflammatory foci induced by the CCl₄ and DEN+CCl₄ administration at the 12 week time point.

Minnelide showed promising results in two models of liver fibrosis. Therapeutic intervention has shown reduction in plasma biochemical markers as well as fibrosis in CCl₄ induced liver fibrosis and DEN+CCl₄ induced liver fibrosis. Additional follow-up with molecular markers by mRNA expression, western blots, collagen quantification, inflammation profiling and oxidative damage parameters can be carried out using models that are well known.

The antifibrotic activity of compounds of formula I can also be evaluated using other known models, such as, for example, a diet-induced mouse model of non-alcoholic fatty liver disease and hepatocellular cancer or a Mouse model of hepatocellular carcinoma with nonalcoholic steatohepatitis using a high-fat, choline deficient diet and intraperitoneal injection of diethylnitrosamine (DEN).

### Example 2 Diet-Induced Obese (DIO) Mouse Model of Non-Alcoholic Steatohepatitis (NASH)

To investigate the effect of 8 weeks of treatment with Minnelide alone and in combination with Elafibranor or Liraglutide in DIO-NASH mouse model, metabolic parameters, hepatic pathology and NAFLD Activity Score (NAS) including Fibrosis Stage data were collected.

### Methods

### Animals

Male C56BL/6JRj mice were obtained from Janvier Labs (Le Genest Saint Isle, France). Mice had ad libitum access to tap water and either regular rodent chow (Altromin 1324, Brogaarden, Hoersholm, Denmark), or a diet high in fat (40%, containing 18% trans-fat), 40% carbohydrates (20% fructose) and 2% cholesterol (AMLN diet; D09100301, Research Diets, New Brunswick, N.J.). C57BL/6JRj mice were fed regular chow as lean chow vehicle control group or AMLN diet as DIO-NASH mice for 35 weeks prior to treatment start.

### Baseline Liver Biopsy

All animals included in the drug treatment experiments underwent liver biopsy for baseline characterization of hepatic parameters and stratified randomization into treatment groups. Mice are anesthetized by inhalation anesthesia using isoflurane (2-3%). A small abdominal incision is made in the midline and the left lateral lobe of the liver is exposed. A cone shaped wedge of liver tissue (approximately 50 mg) is excised from the distal portion of the lobe and fixated in 10% neutral buffered formalin (10% NBF) for histology. The cut surface of the liver is instantly electrocoagulated using bipolar coagulation (ERBE VIO 100 electrosurgical unit). The liver is returned to the abdominal cavity, the abdominal wall is sutured, and the skin is closed with staplers. For post-operative recovery mice will receive carprofen (5 mg/kg) administered subcutaneously on OP day and post-OP day 1 and 2. Animals were allowed to recover for 3˜4 weeks prior to treatment start. Only mice with fibrosis stage ≥1 and steatosis score ≥2 were included in the study for randomization as described previously (Tølbøl, et al. World J Gastroenterol. Jan. 14, 2018; 24(2): 179-194). A stratified randomization into treatment groups is performed according to liver Collagen 1a1 quantification.

### Drug Treatment

Vehicles were 0.5% carboxymethyl cellulose (CMC) with 0.01% Tween-80 (PO dosing) or phosphate-buffered saline with 0.1% bovine serum albumin (SC dosing), administered in a dosing volume of 5 mL/kg. Animals were stratified (n=12-14 per group) and treated for 8 weeks with (1) vehicle (0.5% CMC, PO, QD) and vehicle (saline, SC, QD); (2) Minnelide (0.1 mg/kg, PO, QD) and vehicle (saline, SC, QD), (3) Minnelide (0.1 mg/kg, PO, QD) and liraglutide (0.4 mg/kg, SC, QD); or (4) Minnelide (0.1 mg/kg, PO, QD) and elafibranor (30 mg/kg, PO, QD). A terminal blood sample was collected from the tail vein in non-fasted mice and used for plasma biochemistry. Animals were sacrificed by cardiac puncture under isoflurane anesthesia. Liver samples were processed as described below.

### Biochemical and Histological Analyses

Biochemical and histological analyses were performed as reported previously (Tølbøl, et al. World J Gastroenterol. Jan. 14, 2018; 24(2): 179-194). Plasma analytes included alanine aminotransferase (ALT), aspartate aminotransferase (AST), triglycerides (TG) and total cholesterol (TC). Liver homogenates were analyzed for TG and TC. Paraformaldehyde-fixed liver pre- and post-biopsies were paraffin-embedded, sectioned, and stained with hematoxylin-eosin (Dako, Glostrup, Denmark), Picro-Sirius red (Sigma-Aldrich, Broendby, Denmark), anti-type I collagen (Col1a1; Southern Biotech, Birmingham, Ala.), or anti-galectin-3 (Biolegend, San Diego, Calif., United States). The NAFLD activity score (NAS) and fibrosis staging system was applied to liver pre-biopies and terminal samples (drug treatment experiments) or only terminal samples (disease progression experiment) for scoring of steatosis, lobular inflammation, hepatocyte ballooning, and fibrosis. All histological assessments were performed by a pathologist blind to treatment. Because treatment can affect total liver weight, quantitative data on liver total lipid, galectin-3, Col1a1 content were expressed as whole-liver amounts by multiplying individual terminal liver weight with the corresponding liver lipid concentration (biochemistry data) or percent fractional area (histology data), respectively.

### Results

Metabolic, Biochemical and Histological Changes of DIO-NASH Mice with Vehicle Treatment

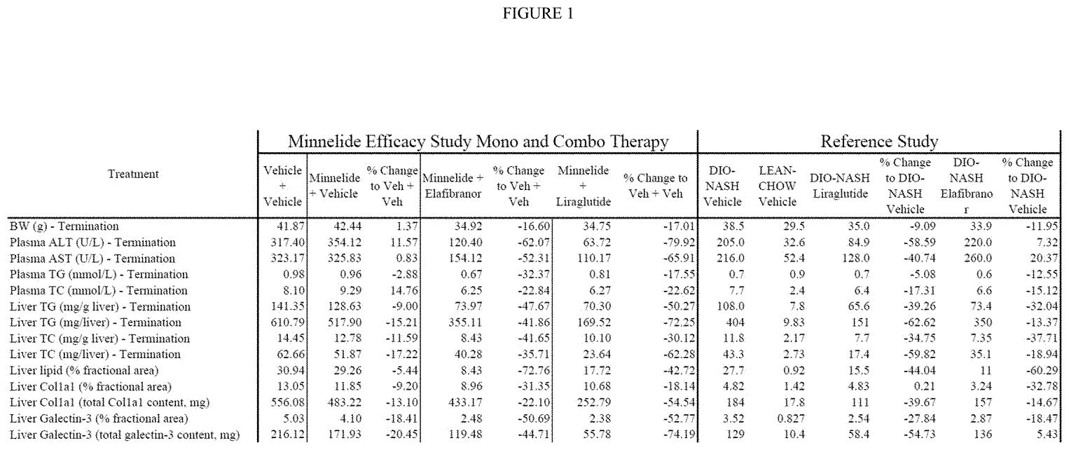

At study termination, DIO-NASH vehicle-treated animals displayed obesity and hepatomegaly in conjunction with increased relative (mg/g) and total levels of liver triglycerides (TG) and total cholesterol (TC) content, as well as elevated levels of plasma TC and liver enzymes ALT/AST (FIG. 1). Steatohepatitis was confirmed histologically (image analysis) by increased relative (%) and total levels of liver steatosis (lipid) and macrophage marker galectin-3 (Gal-3). Furthermore, the fibrotic phenotype was confirmed by increased relative and total levels of liver hydroxyproline (HP), Collagen 1a1 (Col1a1) and alpha-SMA (α-SMA). For histological scoring, 9 of 12 vehicle-treated DIO-NASH animals demonstrated sustained or increased composite NAFLD Activity Score (NAS) (pre-treatment to post-treatment) ranging from 5-7 points and with 3 of 12 animals demonstrating regression from baseline, driven by reduction in lobular inflammation score. Finally, 11 of 12 DIO-NASH vehicle-treated animals demonstrated sustained Fibrosis Stage (pre-treatment to post-treatment), all being F2-F3, with 1 of 12 animal demonstrating regression from baseline. Taken together, the metabolic, biochemical and histological phenotype observed is in accordance with previous findings for the DIO-NASH mouse model.

### Minnelide Lowered Galectin-3 Level in Liver of DIO-NASH Mice

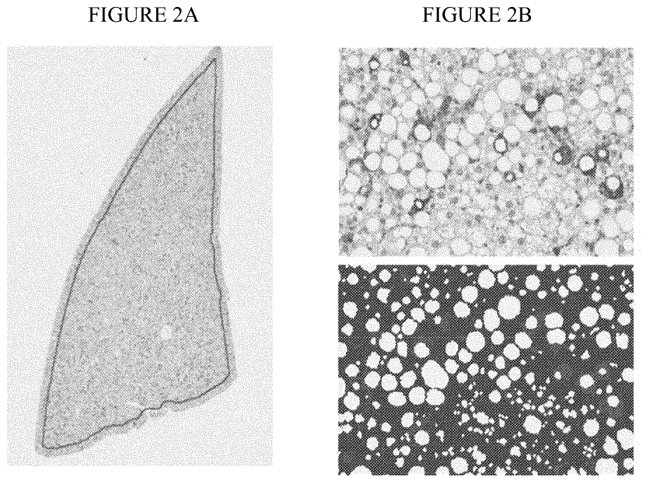

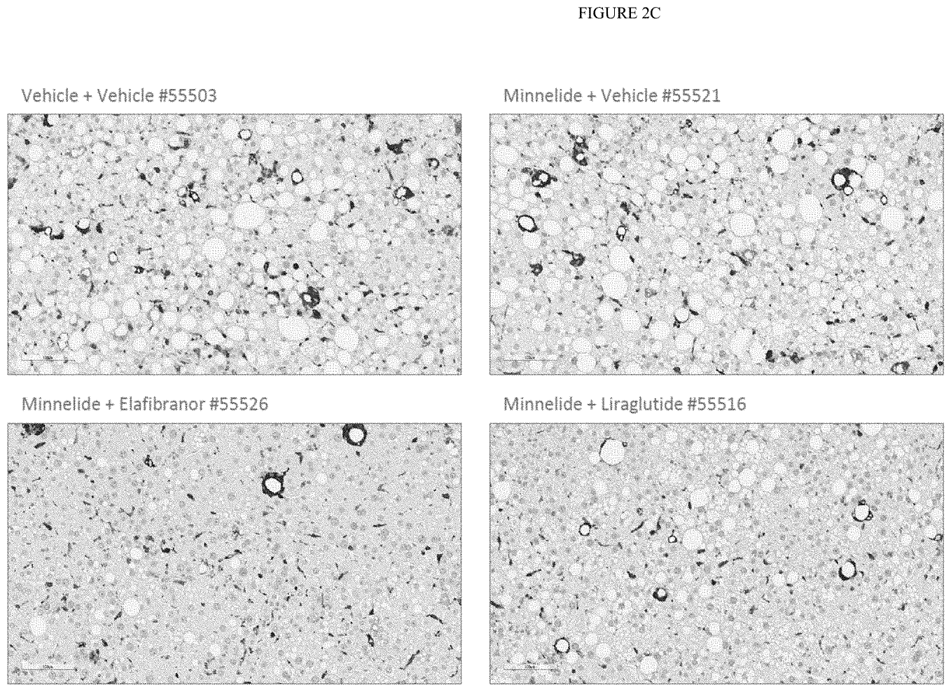

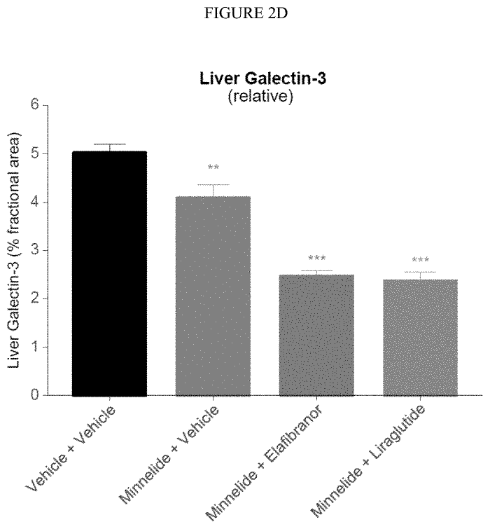

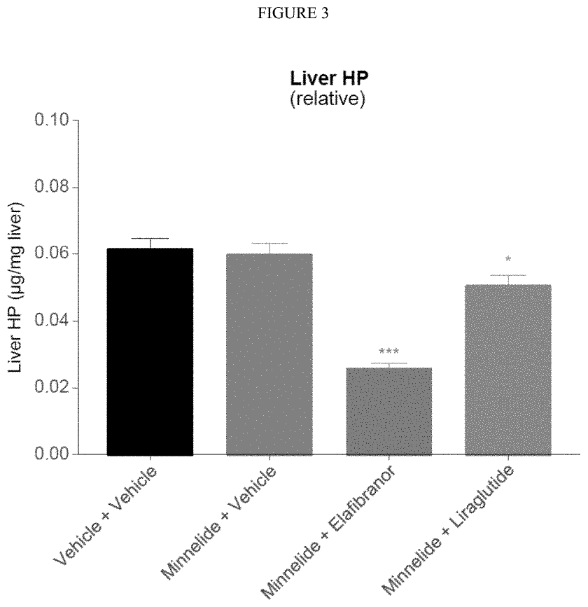

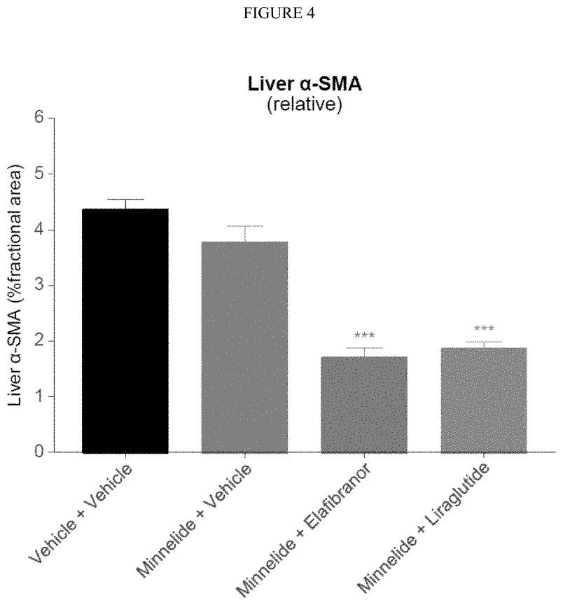

Galectin-3 is a critical protein in the pathogenesis of fatty liver disease and fibrosis. Inhibition of Galectin-3 has shown promising efficacy in protecting from diet-induced NASH in pre-clinical and early clinical studies. The liver Galectin-3 was estimated as fraction of positive Galectin-3 staining area as a percentage of total tissue area (FIG. 2A-B). Compared to vehicle treated control DIO-NASH mice, Minnelide treated group demonstrated 18.41% reduction in liver Galectin-3 (% fractional area) (FIG. 1 and FIG. 2C-D).

### Minnelide and Elafibranor Combination Therapy

Treatment with Minnelide and elafibranor combo-therapy for 8 weeks reduced body weight by approx. 12% from baseline (vehicle-corrected) and concomitantly reduced hepatomegaly, when compared to DIO-NASH vehicle treated animals. In addition, Minnelide and elafibranor treatment reduced plasma levels of ALT/AST/TC/TG and decreased relative and total levels of liver TG/TC content (FIG. 1). Furthermore, Minnelide and elafibranor treatment reduced relative and total levels of liver lipid and Gal-3. For fibrosis, Minnelide and elafibranor treatment decreased relative and total levels of liver hydroxyproline (HP) content, relative levels of liver Col1a1 and relative and total levels of α-SMA. For histological scoring, all Minnelide and elafibranor-treated animals decreased composite NAS (pre-treatment to post-treatment), predominantly driven by reductions in steatosis and lobular inflammation scores. Moreover, Minnelide and elafibranor combination therapy demonstrated an overall trend of enhanced efficacy compared to an elafibranor monotherapy historical reference study published in Tølbøl, et al. World J Gastroenterol. Jan. 14, 2018; 24(2): 179-194 (Reference study of FIG. 1).

### Minnelide and Liraglutide Combination Therapy

Treatment with Minnelide and liraglutide combo-therapy for 8 weeks reduced body weight by approx. 13% from baseline (vehicle-corrected) and concomitantly reduced hepatomegaly, when compared to DIO-NASH vehicle treated animals. In addition, Minnelide and liraglutide treatment reduced plasma levels of ALT/AST/TC and decreased relative and total levels of liver TG/TC content (FIG. 1). Furthermore, Minnelide and liraglutide treatment reduced relative and total levels of liver lipid and Gal-3. For fibrosis, Minnelide and liraglutide treatment decreased relative and total levels of liver HP content, total levels of liver Col1a1 and relative and total levels of α-SMA. For histological scoring, 10 of 13 Minnelide and liraglutide-treated animals decreased composite NAS (pre-treatment to post-treatment), predominantly driven by reductions in lobular inflammation and hepatocellular ballooning scores. Moreover, Minnelide and liraglutide combination therapy demonstrated an overall trend of enhanced efficacy compared to a liraglutide monotherapy historical reference study published in Tølbøl, et al. World J Gastroenterol. Jan. 14, 2018; 24(2): 179-194 (Reference study of FIG. 1).

### Example 3 Preparation of Representative Compounds of Formula I

Minnelide™ (14-O-phosphonooxymethyltriptolide disodium salt) can be prepared as illustrated in the following Scheme.

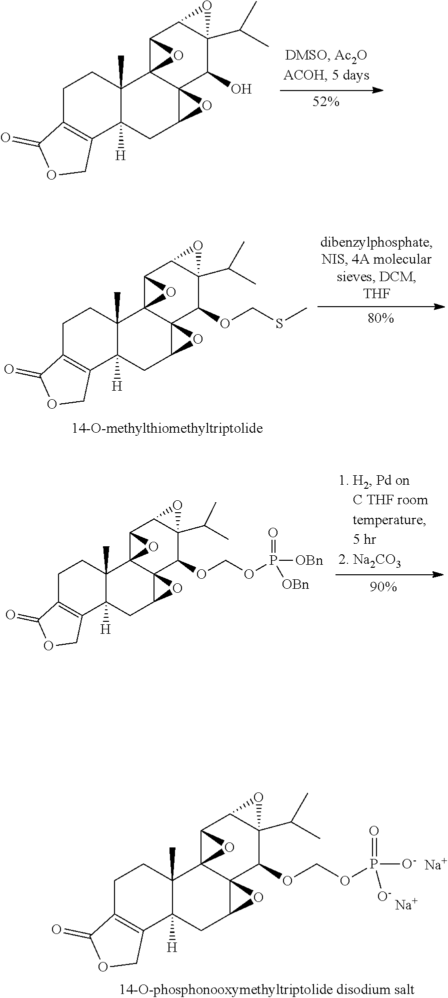 [SMILES: CC(C)[C@]12O[C@H]1[C@@H]1O[C@]13[C@]1(O[C@H]1C[C@H]1C4=C(CC[C@@]13C)C(=O)OC4)[C@@H]2O ; CSCO[C@@H]1[C@@]2(C(C)C)O[C@H]2[C@@H]2O[C@]23[C@]12O[C@H]2C[C@H]1C2=C(CC[C@@]13C)C(=O)OC2 ; CC(C)[C@]12O[C@H]1[C@@H]1O[C@]13[C@]1(O[C@H]1C[C@H]1C4=C(CC[C@@]13C)C(=O)OC4)[C@@H]2OCOP(=O)(OCc1ccccc1)OCc1ccccc1 ; CC(C)[C@]12O[C@H]1[C@@H]1O[C@]13[C@]1(O[C@H]1C[C@H]1C4=C(CC[C@@]13C)C(=O)OC4)[C@@H]2OCOP(=O)([O-])[O-] ; [Na+] ; [Na+]]

### Synthesis of 14-O-phosphonooxymethyltriptolide disodium Salt

To a solution of 14-O-phosphonooxymethyltriptolide dibenzyl ester (50 mg, 0.08 mmol) in tetrahydrofuran (5 mL) was added palladium on carbon (10%, 10 mg). The mixture was stirred at room temperature under hydrogen (1 atm) for a period of 3 hours. The catalyst was removed by filtration through CELITE™, and the filtrate was treated with a solution of sodium carbonate hydrate (8.9 mg in 3 mL water, 0.076 mmol). The tetrahydrofuran was evaporated under reduced pressure and the residual water solution was extracted with ether (3×3 mL). The aqueous layer was evaporated to dryness and the resulting solid was dried overnight in vacuo, washed with ether and again dried in vacuo to provide 14-O-phosphonooxymethyltriptolide disodium salt (35 mg, 90% yield) as a white powder. ¹HNMR (400 MHz, D₂O) δ 0.81 (d, 3H, J=6.8 Hz), 1.00 (d, 3H, J=6.8 Hz), 1.03 (s, 3H), 1.35 (m, 1H), 1.50 (m, 1H), 2.00 (dd, 1H, J₁=14.7 and J₂=13.4 Hz), 2.08-2.61 (m, 4H), 2.85 (m, 1H), 3.63 (d, 1H, J=5.5 Hz), 3.81 (d, 1H, J=3.1 Hz), 3.86 (s, 1H), 4.12 (d, 1H, J=3.1 Hz), 4.92 (m, 2H), 5.07 (m, 2H) ppm; ¹³C NMR (100 MHz, D₂O) δ 12.9, 16.0, 16.3, 16.5, 22.3, 25.5, 28.9, 35.2, 39.8, 55.4, 56.1, 61.0, 61.5, 65.1, 65.5, 71.9, 77.6, 91.7, 123.8, 164.2, 177.3 ppm; HRMS calculated for (C₂₁H₂₆O₁₀P) required m/z [M+1]⁺ 469.1264, found m/z 469.1267.

The intermediate 14-O-phosphonooxymethyltriptolide dibenzyl ester can be prepared as follows.

a. A solution of triptolide (100 mg, 0.29 mmol) in acetic acid (5 mL, 87.5 mmol) and acetic anhydride (1 mL, 10.5 mmol) in DMSO (1.5 mL, 21.4 mmol) was prepared and stirred at room temperature for a period of 5 days to yield 14-O-methylthiomethyltriptolide intermediate. The reaction mixture was then poured into water (100 mL) and neutralized with solid NaHCO₃, added in portions. The mixture was extracted with ethyl acetate (50 mL×3), and the combined organic extract was dried over anhydrous sodium sulfate and concentrated to furnish the product as an oil. Flash silica gel column chromatography (3:2 hexane/ethyl acetate) provided 14-O-methylthiomethyltriptolide in 52% (60 mg) as a white foam. ¹H NMR (400 MHz, CDCl₃) δ 0.82 (d, 3H, J=6.8 Hz), 1.00 (d, 3H, J=6.8 Hz), 1.09 (s, 3H), 1.20 (m, 1H), 1.59 (m, 1H), 1.93 (dd, 1H, J₁=14.7 and J₂=13.4 Hz), 2.19 (s, 3H), 2.10-2.42 (m, 4H), 2.68 (m, 1H), 3.24 (d, 1H, J 5.5 Hz), 3.51 (d, 1H, J=3.1 Hz), 3.67 (s, 1H), 3.79 (d, 1H, J=3.1 Hz), 4.68 (m, 2H), 4.93 (d, 1H, J=11.8 Hz), 5.07 (d, 1H, J=11.8 Hz) ppm; ¹³C NMR (100 MHz, CDCl₃) δ 13.6, 14.8, 16.8, 17.0, 17.1, 23.4, 26.3, 29.5, 35.8, 40.4, 54.5, 55.0, 58.0, 61.5, 63.9, 64.4, 69.9, 75.8, 76.7, 125.5, 160.2, 173.2 ppm; FIRMS calculated for (C₂₂H₂₈O₆SNa) required m/z [M+Na]⁺ 443.1505, found m/z 443.1507.
b. A solution of 14-O-methylthiomethyltriptolide (50 mg, 0.12 mmol) in dry methylene chloride (2 mL) under an N₂ atmosphere was combined with powdered activated 4 Å molecular sieves (50 mg), followed by the addition of a mixture of dibenzylphosphate (40 mg, 0.14 mmol) and N-iodosuccinimide (32 mg, 0.14 mmol) in tetrahydrofuran (2 mL). The reaction mixture was stirred at room temperature for a period of 5 hours, filtered, and diluted with methylene chloride (20 mL). The resulting solution was washed with a solution of sodium thiosulfate (2 mL, 1M solution), a saturated solution of sodium bicarbonate, brine, dried over a sodium sulfate, filtered, and concentrated in vacuo. The oily residue was purified by silica gel flash chromatography (1:2 hexane/ethyl acetate) to give 14-O-phosphonooxymethyltriptolide dibenzyl ester (62 mg, 80% yield) as a white foam. ¹H NMR (400 MHz, CDCl₃) δ 0.72 (d, 3H, J=6.8 Hz), 0.89 (d, 3H, J=6.8 Hz), 1.05 (s, 3H), 1.27 (m, 1H), 1.48 (m, 1H), 1.82 (dd, 1H, J₁=14.7 and J₂=13.4 Hz), 2.03-2.35 (m, 4H), 2.64 (m, 1H), 3.14 (d, 1H, J=5.5 Hz), 3.46 (d, 1H, J 3.1 Hz), 3.65 (s, 1H), 3.76 (d, 1H, J=3.1 Hz), 4.65 (m, 2H), 5.02 (m, 4H), 5.27 (m, 1H), 5.47 (m, 1H), 7.34 (m, 10H) ppm; ¹³C NMR (100 MHz, CDCl₃) δ 13.6, 16.8, 17.0, 23.3, 26.2, 29.62, 29.67, 35.7, 40.3, 54.7, 55.2, 59.3, 61.1, 63.6, 64.0, 69.36, 69.39, 69.42, 69.45, 69.9, 78.2, 92.9, 93.0, 125.5, 127.9, 128.0, 128.6, 135.5, 135.6, 160.1, 173.2 ppm; HRMS calculated for (C₃₅H₃₉O₁₀PNa) required m/z [M+Na]⁺ 673.2179, found m/z 673.2176.

### Example 4 Preparation of Minnelide™ (14-O-Phosphonooxymethyltriptolide Disodium Salt)

Minnelide™ (14-O-phosphonooxymethyltriptolide disodium salt) can also be prepared as illustrated in the following Scheme.

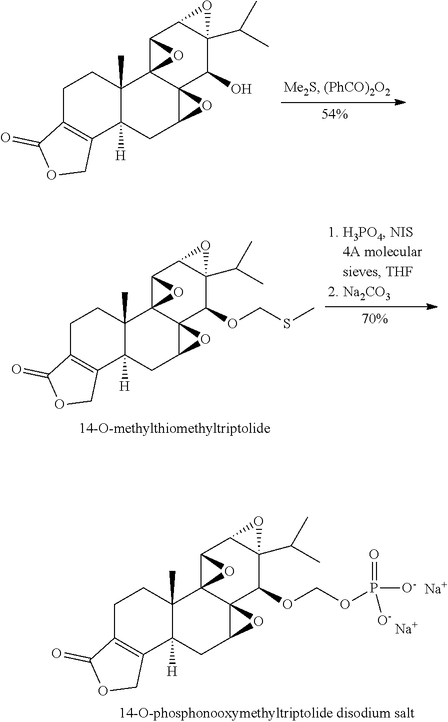 [SMILES: CC(C)[C@]12O[C@H]1[C@@H]1O[C@]13[C@]1(O[C@H]1C[C@H]1C4=C(CC[C@@]13C)C(=O)OC4)[C@@H]2O ; CSCO[C@@H]1[C@@]2(C(C)C)O[C@H]2[C@@H]2O[C@]23[C@]12O[C@H]2C[C@H]1C2=C(CC[C@@]13C)C(=O)OC2 ; CC(C)[C@]12O[C@H]1[C@@H]1O[C@]13[C@]1(O[C@H]1C[C@H]1C4=C(CC[C@@]13C)C(=O)OC4)[C@@H]2OCOP(=O)([O-])[O-] ; [Na+] ; [Na+]]

### Synthesis of 14-O-phosphonooxymethyltriptolide disodium Salt

To a solution containing 14-O-methylthiomethyltriptolide (50 mg, 0.12 mmol), phosphoric acid (82 mg, 0.84 mmol), and molecular sieves (4 Å, 0.45 g) in THF (10 mL) at 0° C. was added N-iodosuccinimide (41 m g, 0.18 mmol), and the mixture was stirred at room temperature for 1 h. The reaction mixture was filtered through Celite, and the solids were washed with THF. The filtrate was treated with 1 M Na₂S₂O₃ until it was colorless and the filtrate was treated with a solution of sodium carbonate (13 mg in 3 mL water, 0.12 mmol). The filtrate was evaporated under reduced pressure and the residual water solution was extracted with ether (3×3 mL). The aqueous layer was evaporated to dryness and the resulting residue was purified by chromatography (C₁₈), eluting with a gradient of 0-100% methanol in water to give 14-O-phosphonooxymethyltriptolide disodium salt (43 mg, 70% yield) as a colorless powder.

The intermediate 14-O-methylthiomethyltriptolide can be prepared as follows.

a. To a solution of triptolide (100 mg, 0.28 mmol) and methyl sulfide (0.16 mL, 2.24 mmol) in acetonitrile (10 mL) at 0° C. was added benzoyl peroxide (0.27 g, 1.12 mmol) in four equal portions over 20 min, and then the mixture was stirred at 0° C. for 1 h and thereafter at room temperature for 1 h. The mixture was diluted with ethyl acetate and washed with 10% Na₂CO₃ and then brine. The organic phase was dried over MgSO₄, filtered, and evaporated. The residue was purified by silica gel flash chromatography (1:1 hexane/ethyl acetate) to furnish 14-O-methylthiomethyltriptolide (63 mg, 54% yield) as a colorless powder.

Minnelide™ (14-O-phosphonooxymethyltriptolide disodium salt) can also be prepared as illustrated in the following Scheme.

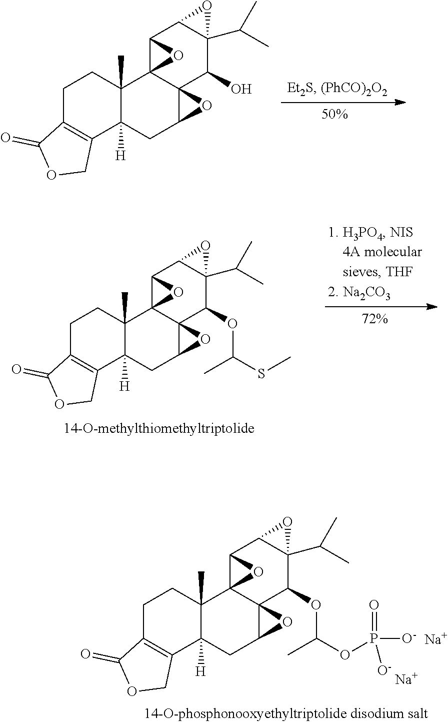 [SMILES: CC(C)[C@]12O[C@H]1[C@@H]1O[C@]13[C@]1(O[C@H]1C[C@H]1C4=C(CC[C@@]13C)C(=O)OC4)[C@@H]2O ; CSC(C)O[C@@H]1[C@@]2(C(C)C)O[C@H]2[C@@H]2O[C@]23[C@]12O[C@H]2C[C@H]1C2=C(CC[C@@]13C)C(=O)OC2 ; CC(O[C@@H]1[C@@]2(C(C)C)O[C@H]2[C@@H]2O[C@]23[C@]12O[C@H]2C[C@H]1C2=C(CC[C@@]13C)C(=O)OC2)OP(=O)([O-])[O-] ; [Na+] ; [Na+]]

### Synthesis of 14-O-Phosphonooxyethyltriptolide disodium Salt

To a solution containing 14-O-methylthioethyltriptolide (52 mg, 0.12 mmol), phosphoric acid (82 mg, 0.84 mmol), and molecular sieves (4 Å, 0.45 g) in THF (10 mL) at 0° C. was added N-iodosuccinimide (41 mg, 0.18 mmol), and the mixture was stirred at room temperature for 1 h. The reaction mixture was filtered through Celite, and the solids were washed with THF. The filtrate was treated with 1 M Na₂S₂O₃ until it was colorless and the filtrate was treated with a solution of sodium carbonate (13 mg in 3 mL water, 0.12 mmol). The filtrate was evaporated under reduced pressure and the residual water solution was extracted with ether (3×3 mL). The aqueous layer was evaporated to dryness and the resulting residue was purified by chromatography (C18), eluting with a gradient of 0-100% methanol in water to give 14-O-phosphonooxyethyltriptolide disodium salt (46 mg, 72% yield) as a colorless powder. ¹H NMR (400 MHz, D₂O) δ 0.68 (d, 3H, J=6.8 Hz), 0.70 (d, 3H, J=6.8 Hz), 1.03 (s, 3H), 1.21 (m, 1H), 1.57 (d, 3H, J=5.3 Hz), 1.58 (m, 1H), 1.94 (dd, 1H, J₁=14.7 and J₂=13.4 Hz), 2.08-2.61 (m, 4H), 2.62 (m, 1H), 3.27 (d, 1H, J=5.5 Hz), 3.45 (d, 1H, J=3.1 Hz), 3.72 (d, 1H, J=3.1 Hz), 3.79 (s, 1H), 4.63 (m, 2H), 6.43 (q, 1H, J=5.3 Hz) ppm; ¹³C NMR (100 MHz, D₂O) δ 13.5, 16.9, 17.0, 17.1, 21.4, 23.5, 26.8, 29.5, 35.9, 40.3, 54.0, 55.1, 59.4, 61.2, 63.6, 64.2, 69.8, 75.8, 76.5, 91.6, 125.6, 164.2, 177.2 ppm; HRMS calculated for (C₂₂H₂₈O₁₀P) required m/z [M+1]⁺ 483.1137, found m/z 483.1134.

The intermediate 14-O-methylthioethyltriptolide can be prepared as follows.

a. To a solution of triptolide (100 mg, 0.28 mmol) and ethyl sulfide (0.24 mL, 2.24 mmol) in acetonitrile (10 mL) at 0° C. was added benzoyl peroxide (0.27 g, 1.12 mmol) in four equal portions over 20 min, and then mixture was stirred at 0° C. for 1 h and then at room temperature for 1 h. The mixture was diluted with ethyl acetate and washed with 10% Na₂CO₃ and then brine. The organic phase was dried over MgSO₄, filtered, and evaporated. The residue was purified by silica gel flash chromatography (1:1 hexane/ethyl acetate) to give 14-O-methylthioethyltriptolide (60 mg, 50% yield) as a colorless powder. ¹H NMR (400 MHz, CDCl₃) δ 0.68 (d, 3H, J=6.8 Hz), 0.70 (d, 3H, J=6.8 Hz), 1.04 (s, 3H), 1.20 (m, 1H), 1.57 (d, 3H, J=5.3 Hz), 1.59 (m, 1H), 1.88 (dd, 1H, J₁=14.7 and J₂=13.4 Hz), 2.19 (s, 3H), 2.06-2.27 (m, 4H), 2.62 (m, 1H), 3.24 (d, 1H, J=5.5 Hz), 3.42 (d, 1H, J=3.1 Hz), 3.70 (d, 1H, J=3.1 Hz), 3.73 (s, 1H), 4.61 (m, 2H), 5.02 (q, 1H, J=5.3 Hz) ppm; ¹³C NMR (100 MHz, CDCl₃) δ 13.6, 14.8, 16.9, 17.0, 17.1, 21.0, 23.5, 26.4, 29.6, 35.8, 40.5, 54.0, 55.2, 59.4, 61.3, 63.7, 64.2, 69.9, 75.8, 76.7, 125.6, 160.2, 173.2 ppm; HRMS calculated for (C₂₃H₃₀O₆SNa) required m/z [M+Na]⁺ 457.1763, found m/z 457.1765.

Minnelide™ (14-O-phosphonooxymethyltriptolide disodium salt) can also be prepared as illustrated in the following Scheme.

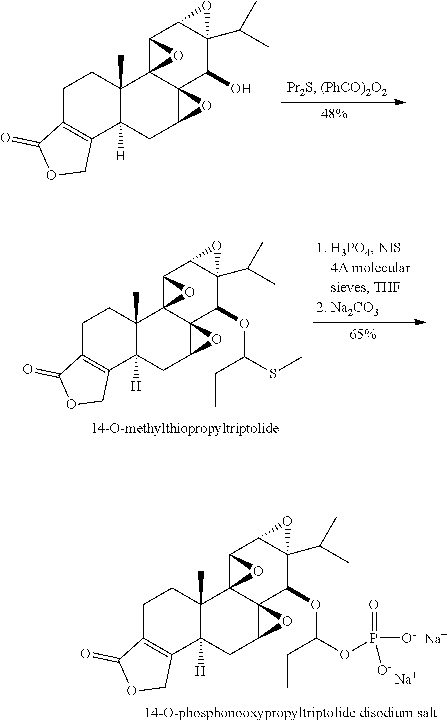 [SMILES: CC(C)[C@]12O[C@H]1[C@@H]1O[C@]13[C@]1(O[C@H]1C[C@H]1C4=C(CC[C@@]13C)C(=O)OC4)[C@@H]2O ; CCC(O[C@@H]1[C@@]2(C(C)C)O[C@H]2[C@@H]2O[C@]23[C@]12O[C@H]2C[C@H]1C2=C(CC[C@@]13C)C(=O)OC2)SC ; CCC(O[C@@H]1[C@@]2(C(C)C)O[C@H]2[C@@H]2O[C@]23[C@]12O[C@H]2C[C@H]1C2=C(CC[C@@]13C)C(=O)OC2)OP(=O)([O-])[O-] ; [Na+] ; [Na+]]

### Synthesis of 14-O-Phosphonooxypropyltriptolide disodium Salt

To a solution containing 14-O-methylthiopropyltriptolide (54 mg, 0.12 mmol), phosphoric acid (82 mg, 0.84 mmol), and molecular sieves (4 Å, 0.45 g) in THF (10 mL) at 0° C. was added N-iodosuccinimide (41 mg, 0.18 mmol), and the mixture was stirred at room temperature for 1 h. The reaction mixture was filtered through Celite, and the solids were washed with THF. The filtrate was treated with 1 M Na₂S₂O₃ until it was colorless and the filtrate was treated with a solution of sodium carbonate (13 mg in 3 mL water, 0.12 mmol). The filtrate was evaporated under reduced pressure and the residual water solution was extracted with ether (3×3 mL). The aqueous layer was evaporated to dryness and the resulting residue was purified by chromatography (C18), eluting with a gradient of 0-100% methanol in water to provide 14-O-phosphonooxypropyltriptolide disodium salt (43 mg, 65% yield) as a colorless powder. ¹H NMR (400 MHz, D₂O) δ 0.66 (d, 3H, J=6.8 Hz), 0.68 (d, 3H, J=6.8 Hz), 0.99 (t, 3H, J=5.3 Hz), 1.03 (s, 3H), 1.20 (m, 1H), 1.53 (m, 1H), 1.90 (dd, 1H, J₁=14.7 and J₂=13.4 Hz), 2.04-2.66 (m, 4H), 2.65 (m, 3H), 3.27 (d, 1H, J=5.5 Hz), 3.49 (d, 1H, J=3.1 Hz), 3.71 (d, 1H, J=3.1 Hz), 3.78 (s, 1H), 4.69 (m, 2H), 6.31 (q, 1H, J=5.3 Hz) ppm; ¹³C NMR (100 MHz, D₂O) δ 7.55, 13.5, 16.2, 16.9, 17.2, 20.8, 23.2, 26.1, 28.4, 34.7, 38.5, 54.1, 55.0, 59.0, 61.3, 62.5, 63.9, 68.5, 75.4, 76.4, 91.9, 125.7, 160.1, 174.5 ppm; HRMS calculated for (C₂₃H₂₉O₁₀P) required m/z [M+1]⁺ 497.1294, found m/z 497.1292.

The intermediate 14-O-methylthiopropyltriptolide can be prepared as follows.

a. To a solution of triptolide (100 mg, 0.28 mmol) and propyl sulfide (0.32 mL, 2.24 mmol) in acetonitrile (10 mL) at 0° C. was added benzoyl peroxide (0.27 g, 1.12 mmol) in four equal portions over 20 min, and the mixture was stirred at 0° C. for 1 h and then at room temperature for 1 h. The mixture was diluted with ethyl acetate and washed with 10% Na₂CO₃ and then brine. The organic phase was dried over MgSO₄, filtered, and evaporated. The residue was purified by silica gel flash chromatography (1:1 hexane/ethyl acetate) to give 14-O-methylthiopropyltriptolide (60 mg, 48% yield) as a colorless powder. ¹H NMR (400 MHz, CDCl₃) δ 0.65 (d, 3H, J=6.8 Hz), 0.67 (d, 3H, J=6.8 Hz), 0.99 (t, 3H, J=5.3 Hz), 1.01 (s, 3H), 1.20 (m, 1H), 1.59 (m, 1H), 1.88 (dd, 1H, J₁=14.7 and J₂=13.4 Hz), 2.18 (s, 3H), 2.01-2.26 (m, 4H), 2.62 (m, 3H), 3.24 (d, 1H, J=5.5 Hz), 3.42 (d, 1H, J=3.1 Hz), 3.70 (d, 1H, J=3.1 Hz), 3.73 (s, 1H), 4.61 (m, 2H), 5.03 (q, 1H, J=5.3 Hz) ppm; ¹³C NMR (100 MHz, CDCl₃) δ 7.68, 13.5, 14.6, 16.2, 17.0, 17.2, 21.4, 23.2, 26.1, 28.9, 34.7, 39.5, 54.1, 55.6, 59.0, 61.3, 63.5, 64.0, 69.5, 75.1, 76.4, 125.1, 160.9, 173.5 ppm; FIRMS calculated for (C₂₄H₃₂O₆SNa) required m/z [M+Na]⁺ 471.1920, found m/z 471.1918.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for treating fibrosis, nonalcoholic fatty liver disease (NAFLD) or nonalcoholic steatohepatitis (NASH) in an animal, comprising administering to the animal, a compound of formula I: 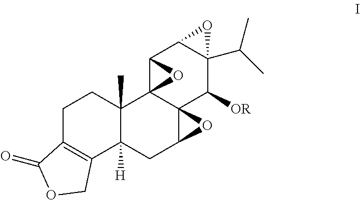
wherein:
R is H or (CR¹R²O)ₙP(O)(OH)₂;
each R¹ is independently H, (C₁-C₆)alkyl, aryl(C₁-C₆)alkyl-, (C₃-C₆)cycloalkyl or aryl; and each R² is independently H, (C₁-C₆)alkyl, aryl(C₁-C₆)alkyl-, (C₃-C₆)cycloalkyl or aryl; or R¹ and R² together with the atom to which they are attached form a (C₃-C₇)cycloalkyl; wherein any alkyl or cycloalkyl of R¹ or R² may be optionally substituted with one or more (e.g. 1, 2, 3, 4 or 5) groups selected from halo, (C₁-C₆)alkoxy and NR^{a}R^{b} and wherein any aryl of R¹ or R² may be optionally substituted with one or more (e.g. 1, 2, 3, 4 or 5) groups selected from halo, (C₁-C₆)alkyl, (C₁-C₆)alkoxy, NR^{a}R^{b}, nitro and cyano;
R^{a} and R^{b} are each independently selected from H, (C₁-C₆)alkyl, (C₃-C₆)cycloalkyl and aryl; or R^{a} and R^{b} together with the nitrogen to which they are attached form a pyrrolidino, piperidino, piperazino, azetidino, morpholino, or thiomorpholino; and
n is 1, 2 or 3;
or a pharmaceutically acceptable salt thereof.

2. The method of claim 1, which is a method of treating nonalcoholic fatty liver disease.

3. The method of claim 1, which is a method of treating nonalcoholic steatohepatitis.

4. The method of claim 1, which is a method of treating liver fibrosis.

5. The method of claim 1, which is a method of treating pulmonary fibrosis.

6. The method of claim 1 wherein a pharmaceutically acceptable salt of formula Ia: 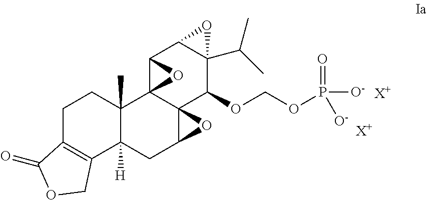
wherein each X⁺ is independently a pharmaceutically acceptable organic cation or a pharmaceutically acceptable inorganic cation is administered.

7. The method of claim 1, wherein 14-O-phosphonooxymethyltriptolide disodium salt, 14-O-phosphonooxyethyltriptolide disodium salt or 14-O-phosphonooxypropyltriptolide disodium salt is administered.

8. The method of claim 1, wherein 14-O-phosphonooxymethyltriptolide disodium salt is administered.

9. The method of claim 1 further comprising administering a GLP-1 agonist or a PPAR agonist to the animal.

10. The method of claim 1 further comprising administering liraglutide to the mammal.

11. The method of claim 1 further comprising administering elafibranor or a salt thereof to the mammal.

12. A pharmaceutical composition comprising: a) a compound of formula I or a pharmaceutically acceptable salt thereof as described in claim 1, b) a therapeutic agent, and c) a pharmaceutically acceptable diluent or carrier.

13. The composition of claim 12, wherein the therapeutic agent is selected from the group consisting of insulin sensitizing agents, thiazolidineones, vitamin E, ursodeoxycholic acid, omega-3 fatty acids, galectin-3 inhibitors (e.g., GR-MD-02), and statins.

14. The composition of claim 12, wherein the therapeutic agent is a GLP-1 agonist or a PPAR agonist.

15. The composition of claim 12, wherein the therapeutic agent is liraglutide.

16. The composition of claim 12, wherein the therapeutic agent is elafibranor or a salt thereof.

17. A pharmaceutical composition comprising: a) 14-O-phosphonooxymethyltriptolide disodium salt, b) a therapeutic agent, and c) a pharmaceutically acceptable diluent or carrier.

18. The composition of claim 17, wherein the therapeutic agent is selected from the group consisting of insulin sensitizing agents, thiazolidineones, vitamin E, ursodeoxycholic acid, omega-3 fatty acids, galectin-3 inhibitors (e.g., GR-MD-02), and statins.

19. The composition of claim 17, wherein the therapeutic agent is a GLP-1 agonist or a PPAR agonist.

20. The composition of claim 17, wherein the therapeutic agent is liraglutide or elafibranor or a salt thereof.
